(19) United States
(12) Patent Application Publication
(10) Pub. No.: US 2008/0188859 A1
(43) Pub. Date: Aug. 7, 2008

(54) Surgical instrument for implanting a wire, preferably into a bone

(76) Inventors: Cliff-Georg Reitzig, Weilheim-Rietheim (DE); Stephan Eckhof, Weilheim-Rietheim (DE); Thomas Feldhaus, Tuttlingen (DE)

Correspondence Address: COLLARD & ROE, P.C., 1077 NORTHERN BOULEVARD, ROSLYN, NY, 11576, US

(21) Appl. No.: 11/999,535

(22) Filed: Dec. 6, 2007

(30) Foreign Application Priority Data
Dec. 6, 2006 (DE) 20 2006 018 587.9

(51) Int. Cl. A61B 17/58; A61B 17/00

(52) U.S. Cl. 606/103; 606/205

(57) ABSTRACT

A surgical instrument for implanting a wire, preferably into a bone, can be operated with one hand and thus acts as an extension of a surgeon's arm. The instrument is made up of a receiving element and a clamp part and also of a grip element. The grip element has two grip parts, the receiving element including the first grip part, and the clamp part including the second grip part. The receiving element has a recess for at least partial support of the wire, and the clamp part is mounted on the receiving element, so as to be able to move in rotation about a bearing point, and includes a clamp element which engages in the recess and is designed in such a way that, when the grip parts are pressed together, the clamp part presses the wire against the support.

### CROSS-REFERENCE TO RELATED APPLICATIONS

This application claims convention priority under 35 U.S.C. §119 from German Utility model DE 20 2006 018 587.9 filed on Dec. 6, 2006.

### BACKGROUND TO THE INVENTION

1. Field of the Invention

The invention relates to a surgical instrument. In particular, the invention relates to a surgical instrument for manipulating a straight or bent surgical wire for use in an intraoperative procedure.

Surgical wires, for example Kirschner wires (or K-wires) are generally used for fixing bone fractures. In a first fixing step, a bore is formed in the bone fragment to be fixed and one is formed in the adjacent bone. In a further step, the wire is guided through these two bores and, for example by twisting or connection to further fixing elements, is positioned in such a way that the fracture is fixed and a fusion of the bone is again possible by this means.

Various tools are needed to perform this procedure. First, aids are provided for initially bending the wire at its free end, such that it can be introduced into a bore provided in the bone. Corresponding bending tools are provided for this purpose. Further auxiliary tools are used to introduce the wire into the two bores. When the wire has been guided through both bores, it is known to use wire pliers in order to bend the end protruding from the bone and, if appropriate, cut said end off. At its end near the bone, the wire is thereby fixed in such wire pliers and, when the hand grips are pressed further together, is correspondingly bent (tension band osteosynthesis). In classical tension band osteosynthesis, the end of the Kirschner wire protruding from the bone is shortened to a defined length and then bent round. By bending the end of the wire round and pressing it into the bone, migration of the wire within the bone is prevented, as also is rotation of the wire about its longitudinal axis. If bone plates are additionally used for supporting the fracture, then further instruments such as cutting pliers, bending pliers and the like have to be used.

The wire thicknesses most commonly used are between 0.8 and 2 mm. The use of the corresponding diameters depends very much on the stressing of the individual bone fragments. Strongly stressed areas, for example the hips, require larger diameters for wire fixing, whereas correspondingly thinner wires are used in less stressed bone fragments.

Inserting the wire into a bore provided in a bone is generally done using customary surgical instruments, for example pliers. To do this, the operating surgeon grips the surgical wire with the clamping jaws of the pliers and guides the wire into the bore. When he has almost reached the outer wall of the bone with the clamping jaws, the pliers are released and the wire has to be clamped again at a further site spaced apart from the bone. By application of the clamping force, it is possible, by advancing the pliers in the direction of the bone, to insert the wire further into the bore.

2. The Prior Art

For inserting the wire, for example a Kirschner wire (K-wire), customary surgical instruments are known. They are designed like pliers and are shaped such that the wire is clamped between two clamping jaws. The clamping jaws are connected in the manner of pliers to grip elements such that, by pressing together the grip parts forming the grip element, a clamping force is exerted on the wire. The clamping jaws themselves are generally flat, with the result that it is not absolutely necessary to ensure an exact positioning of the clamping jaws in order to effect an advance movement inside the bore in the bone.

DE 102004033633 A (STRYKER LEIBINGER GMBH) 02.16.2006 discloses a surgical instrument for manipulating a bent surgical wire, with two jaws that can be moved relative to each other to generate a clamping force, at least one of the jaws having a wire-receiving means.

U.S. Pat. No. 5,116,340 A (SONGER ROBERT J (US); SONGER MATTHEW N (US)) May 26, 1992 discloses a surgical securing instrument shaped like pliers, with two jaws that can be moved relative to each other to generate a clamping force, the jaws having mutually opposite receiving means for crimping surgical wires.

WO 96/39976 A (BURKE DENNIS W (US)) Dec. 19, 1996 discloses an instrument in the form of pliers for tightening and crimping surgical wire in orthopedic surgery, the wire being guided in receiving means in the clamping jaws.

U.S. Pat. No. 5,314,431 A (GRAZIANO THOMAS A) May 24, 1994 discloses an instrument in the shape of pliers for bending a protruding surgical wire, its clamping jaws having an angled receiving means for the surgical wire.

DE 202005006132 U (INTERCUS GMBH (DE)) Jul. 28, 2005 discloses a targeting device for Kirschner wires, comprising a plate on which a holder grip is arranged and on which at least two guide sleeves are arranged relative to each other such that the axes intersect outside a fracture.

### SUMMARY OF THE INVENTION

The object of the invention is to make available a surgical instrument for improved manipulation of a surgical wire. In particular, the surgical instrument is intended to be suitable for correctly inserting a surgical wire into a bore already provided inside a bone.

This object is achieved by a surgical instrument consisting essentially of a receiving element at least for receiving the wire to be applied, a clamp part at least for clamping the wire, and a grip element for holding the surgical instrument. According to the invention, the surgical instrument is one in which the grip element consists of two grip parts, the receiving element comprising the first grip part and the clamp part comprising the second grip part, the receiving element has a recess for at least partial support of the wire, and the clamp part is mounted on the receiving element, so as to be able to move in rotation about a bearing, and comprises a clamp element which engages in the recess and is designed in such a way that, when the grip parts are pressed together, the clamp part presses the wire against the support.

### ADVANTAGES OF THE INVENTION

In a first step for wire osteosynthesis in intraoperative fracture treatment in which the bone fragments are connected to one another with wires, in most cases called Kirschner wires or K-wires, it is necessary first to bend the free end of the wire. Angles of between 5 and 25 degrees are generally provided to permit insertion into a bore that has been formed inside a bone. Alternatively, provision is also made to completely dispense with said bending in the first instance, such that the surgical wire to be implanted is fully stretched and its length is such that it is arranged completely within the surgical instrument according to the invention. Consequently, no free end of the wire protrudes from the instrument.

The invention does not therefore follow the path of using a surgical instrument designed like pliers, but instead to initially make available a receiving element which preferably completely receives the surgical wire to be applied. It is received completely by means of the receiving element, at least in a part area thereof, having a recess in which part of the elongate surgical wire is received. This affords the possibility of the surgical wire that is to be applied being mounted in the receiving element, specifically in such a way that it cannot change its corresponding position inside the receiving element. In a design like pliers, by contrast, the wire to be applied would ultimately be arranged freely between the clamping jaws, in which case, when the pliers-like instrument is pressed together, the clamping jaws at least partially grip the wire that is to be applied. The remaining part of the surgical wire is free and openly accessible. There is a considerable risk of injury when operating with such a design.

The further considerable advantage of the invention is that, relative to the inherently fixed receiving element, which also provides means for receiving the surgical wire to be applied, a movably mounted clamp part is also provided. The clamp part is designed in such a way that it likewise comprises a grip part of the overall grip element and, at the end remote from the grip part, is provided with clamp elements that are designed to clamp the wire inside the receiving element.

The relation of clamp part to receiving element is such that, by pressing the grip parts together with one hand, the surgical wire is clamped inside the receiving element, so as to allow the wire to be driven forward inside the bore. If at least one grip part, either that of the clamp part or that of the receiving element, is released again, the clamp part opens and the clamping action reduces. In this way it is possible for the surgeon to remove the surgical instrument further from the bone and for the surgical wire, however, to remain in its already inserted position. The reason for this is that the static friction with respect to the wire in the bore is greater than that inside the receiving element. By this means, more wire is in turn freed from the surgical instrument, such that further implantation of the wire inside the bore is possible.

Thus, as has been shown, an important feature of this invention is that the surgical instrument for applying a surgical wire can be used with one hand. It suffices for the receiving element to be held in the hand and for the clamp part to be moved up and down relative to it using the fingers or hand, such that, firstly, a clamping action is obtained, and secondly, however, the release of the wire is also obtained.

In a particularly preferred embodiment, the surgical instrument according to the invention consists of two structural parts, namely the receiving element for receiving the wire that is to be applied, and the clamp part for clamping the wire. These two parts are coupled to one another in the manner of pliers. The receiving element advantageously comprises pins, which are provided as bearings for connection of the clamp part thereto. The clamp part is preferably designed in such a way that it has eyelet-like openings, which are designed such that the clamp part can be clipped onto the pins arranged on the receiving element, and can be pivoted about these pins.

The receiving element and pins are preferably designed as a one-piece component, such that it is possible to produce the receiving element in one injection molding operation and to produce the clamp part in a further injection molding operation.

The clamp part itself is designed in such a way that, with respect to the longitudinal extent of the clamp part, most of it is formed by a grip part. The clamp surface is arranged extending away from the bearing (away from the grip part), said clamp surface preferably having a suitable recess adapted to the diameter of the surgical wire that is to be applied. The clamp part and clamp surface are preferably in one piece, such that they too can be produced in a single injection molding operation.

In one development, the clamp surfaces of the clamp part are lined with a metal element. The metal element, which encloses the clamp surface, can either be applied at a later stage or is already fitted in said injection molding operation. By means of this metal element, the clamping force can be further increased and the wear that occurs in this area can be reduced.

In one development, the clamp surface is profiled. This means that the clamp surface can be corrugated, with the aim of increasing the frictional force between the clamp surface and the wire that is to be clamped. Alternatively, or in addition, a profile, for example a notch, can be provided in the direction of the longitudinal extent of the wire, such that the wire to be clamped works its way into the notch during the clamping procedure and is additionally clamped therein. The notches can be of different shapes.

In another advantageous embodiment, additional clamping or guiding means are similarly provided in the area of the recess in the receiving element and are suitable for receiving the surgical wire that is to be applied. The recess, for example in the form of a channel, is in this case designed in such a way that it can receive surgical wires of different diameters.

These described profiles also have the advantage that, by this measure, the wire that is to be applied is exactly positioned inside the receiving element, such that, when external forces act on the free end of the wire, no displacement of the wire inside the instrument takes place.

This ensures that the surgical instrument according to the invention is suitable for receiving surgical wires, for example Kirschner wires of small diameter, for example 0.8 mm to 2 mm, in a single device, and for applying them. However, the invention is not restricted to receiving these stated diameters. Instead, the scope of protection extends, in respect of the wire, to sizes and cross sections that are used for fixing bone. Many different diameters and also different cross sections are suitable for this, however.

In one development of the invention, bending means are also provided in the receiving element. These bending means are designed such that, for example, a free end of a surgical wire can be suitably bent, such that the bent part adopts a defined angle with respect to the remaining, elongate part. A control means likewise provided in the receiving element and in the form of a gage can be used to control and dimension the generated angle.

In a preferred embodiment of the instrument, the whole instrument is made of metal. By virtue of this choice of material, it can be sterilized.

In another embodiment, the product is made of plastic. It can thus be used as a disposable product. To achieve a good clamping force in a plastic design, the clamp area is metallized or provided with a metal plate.

Provided at the free end of the grip element, there is an auxiliary means whose property is such that the two clamp parts can be pressed together only when the surgical wire does not protrude beyond the free end of the surgical instrument at the grip end. A flap-like configuration extending from the clamp part in the direction of the free end of the receiving element is designed such that, when the surgical instrument is pressed together, this free flap also covers the free end of the receiving element. In this way, the free end of the bore in which the wire for application is guided is also covered, thus ensuring that the wire does not emerge in an undesired way at the grip end. This means that it is not possible for the surgical wire to protrude beyond the grip end, thus avoiding injury to the surgeon's hand.

### Illustrative Embodiment

### BRIEF DESCRIPTION OF THE DRAWINGS

Other details and advantages of the invention will become clear from the following description of a preferred illustrative embodiment of the invention and from the figures, in which:

FIG. 1 shows a perspective view of a surgical instrument according to the invention;

FIG. 2 shows a perspective view of a surgical instrument according to the invention;

FIG. 3 shows a schematic representation of the clamping principle of the surgical instrument according to FIGS. 1 and 2;

FIG. 4 shows a cross section through the surgical instrument according to FIGS. 1 or 2, with a surgical wire that has been received but is not clamped (in contrast to FIGS. 1 or 2);

FIG. 5 shows a cross section through the surgical instrument according to FIGS. 1 or 2, with a surgical wire that has been received and is clamped (in contrast to FIG. 1);

FIGS. 6a and b show a schematic representation of a cross section through the surgical instrument according to FIG. 5 along a line VI-VI in order to illustrate the clamping action of different diameters of surgical wires;

FIGS. 7a and b show a representation of the function of the bending device provided in the receiving element;

FIG. 8 shows a schematic representation of the use of the surgical instrument according to the preceding figures.

### DETAILED DESCRIPTION OF A PREFERRED EMBODIMENT

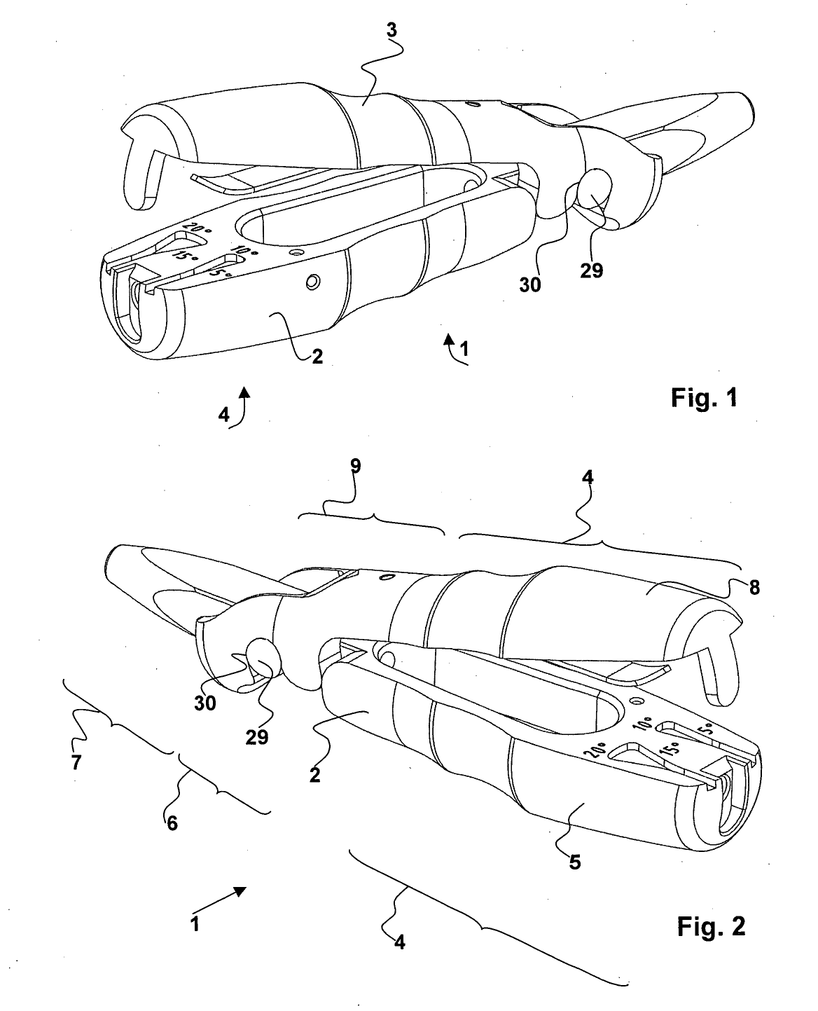

An illustrative embodiment of a surgical instrument according to the invention is described below by way of example and with reference to FIGS. 1-8.

FIGS. 1 and 2 show an overall perspective view of the surgical instrument 1 according to the invention for manipulating and implementing a surgical wire, preferably a Kirschner wire. The wire is not shown in the illustrative embodiment shown in FIGS. 1 and 2.

The surgical instrument 1 comprises two structural parts, namely a receiving element 2 and a clamp part 3. The surgical instrument itself comprises a grip element 4 with which the surgical instrument can be operated with one hand.

The receiving element 2 essentially comprises three functional areas. The first functional area is that of the grip element 4 and is formed by a grip part 5. Adjacent to the grip part 5 there is a bearing point 6, and the bearing point 6 in turn is adjoined by a guiding and positioning element 7. The bearing point 6 comprises pins 29 that interact with a bearing element 9 of the clamp part 3.

The clamp part 3 also has three functional areas. The first functional area is that of the grip element 4 and is formed by a grip part 8. Adjacent to the grip part 8 there is in turn a bearing element 9, which interacts with the bearing point 6 of the receiving element 2. The bearing element 9 is adjoined by a clamp element 10 that comprises the clamp part.

Both structural parts, namely the receiving element 2 and the clamp part 3, are designed as one-piece components and form the surgical instrument 1 by interacting via the bearing point 6 and via the bearing element 9.

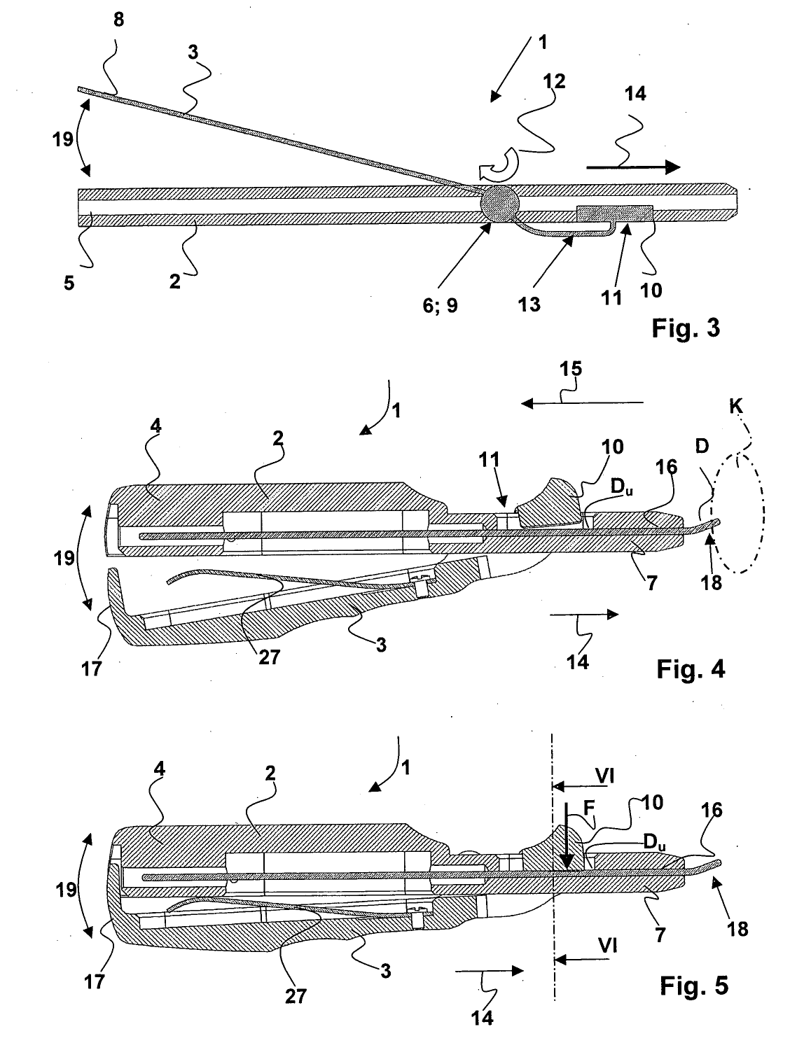

FIG. 3 shows the principle of the surgical instrument 1 according to the invention. The wire to be applied is mounted in the receiving element 2. The clamp part 3 is coupled to the receiving element via bearing points 6, 9. At its end directed away from the grip part 8, the clamp part 3 comprises the clamp element 10, which effects clamping in arrow direction 13 when the clamp part 3 is pivoted in arrow direction 12. The clamp element 10 in this case passes through a recess 11 provided in the receiving element 2. By means of said clamping, the wire is fixed in and counter to the arrow direction 14. In this situation, which is achieved by the two grip parts 5, 8 being pressed together in arrow direction 19, the wire can be applied.

### Mode of Operation

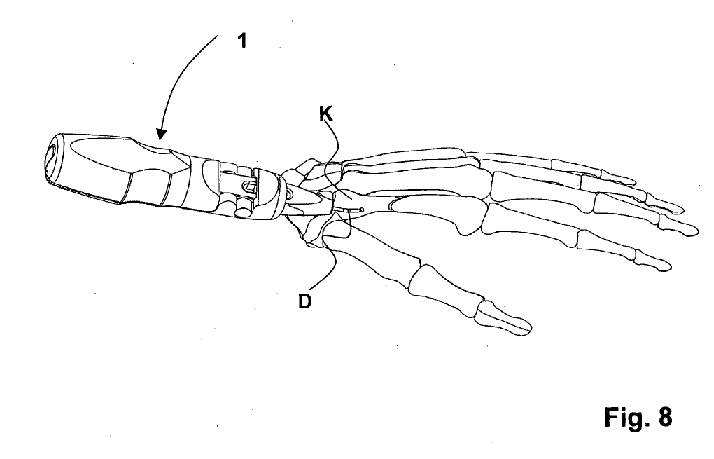

The mode of operation of the surgical instrument 1 is illustrated in FIGS. 4 and 8.

FIG. 4 shows that the wire D has been inserted into the receiving element 2. On introduction into the surgical instrument 1 in arrow direction 15, the wire D is guided in via the guiding and positioning element 7, which comprises a bore 16, and is pushed as far as a limit stop 17, which is provided in the area of the grip element 4. To ensure that the wire D strikes the limit stop 17, the grip element 4 is lightly pressed together such that no clamping as yet takes place.

An alternative for introduction of the wire D involves the latter being guided in rearward, i.e. in the opposite direction to the guiding and positioning element 7, until one end of the wire D is positioned completely inside the receiving element 2 and the grip element 4 can be pressed together.

Depending on the particular use, the surgical wire that is to be applied can also be designed bent in its start area 18, as is shown by way of example in FIG. 4. To insert the surgical wire D into the receiving element 2, it is necessary that the two grip parts 4 and 5 are spaced apart from each other in such a way that the clamp element 10 does not bear on the outer circumference Dᵤ. In this situation, there is no clamping action generated by the clamp part 3.

For applying the surgical wire D, the situation shown in FIG. 5 has to be reached. This is reached by the two grip parts 5, 8 being pressed together in arrow direction 19, such that the clamp element 10 presses directly on the outer circumference Dᵤ of the wire D. This pressing generates a force F perpendicular to the longitudinal extent of the wire D, and the wire D is thus pressed against the inside wall of the bore 16 of the receiving element 2 and is also clamped there, such that a displacement of the wire D in and counter to the arrow direction 14 is no longer possible. With this clamped state, the wire D can be applied.

When the free front end of the guiding and positioning element 7 comes into the immediate area of the wall of the bone K indicated only by broken lines in FIG. 4, the operator has to release the two grip parts 5 and 8, such that the situation shown in FIG. 4 is reached. By virtue of the counteracting force that occurs when the two grip parts are pressed together, the operator reaches the situation shown in FIG. 4 only by opening the surfaces of his hand slightly further, such that the two grip parts 5, 8 are able to spring open spontaneously counter to the arrow direction 19. It is not necessary here for the operator to completely let go of the grip element 4, and instead it suffices to slightly open the fingers alone and pull the surgical instrument 1 away from the bone K counter to the arrow direction 14. When the distance from the outer wall of the bone K is again sufficiently great, the operator can once again bring about the desired clamping action and apply the wire D further. The operator repeats this procedure until the wire D is completely applied.

In a particularly preferred illustrative embodiment, the opening of the two grip parts 5 and 8 is assisted by a spring element 27, as is shown in FIGS. 1, 2, 4 and 5. By pressing the two grip parts 5 and 8 closed in arrow direction 19, energy is stored in the spring element, which energy is then released when the grip parts 5 and 8 are let go.

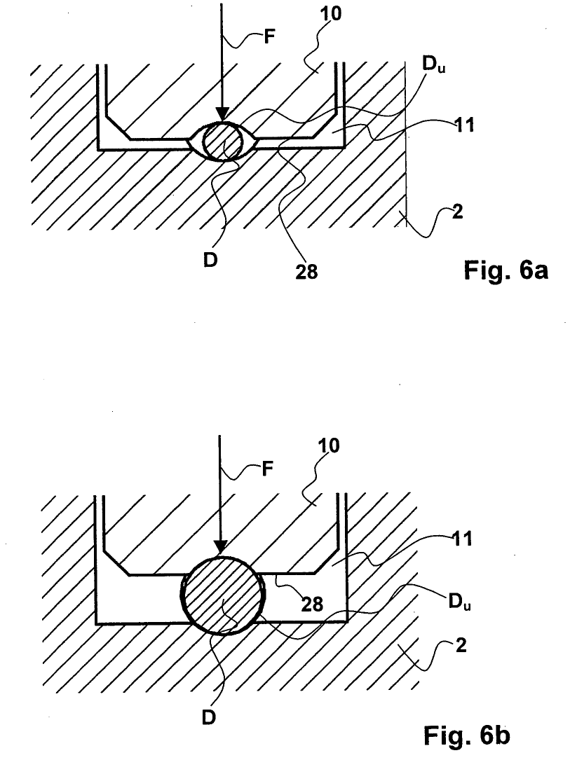

FIGS. 6a and 6b each show a schematic cross section through the surgical instrument 1 shown in FIG. 5. These show a clamping action that is generated by the clamp element 10 pressing against the outer circumference Dᵤ of the wire D and thus pressing the wire D against the receiving element 2. This is effected by a clamping force F, as is shown in FIGS. 6a and 6b.

The clamp parts 10 and also part of the recess 11 are configured in such a way that different diameters of surgical wires D can be received. FIG. 6a shows how a surgical wire D with a small diameter can be received and how a corresponding clamping force F can be generated. FIG. 6b shows a surgical wire D in which the diameter of the wire D is considerably greater. The illustrated clamp surfaces 28 of the clamp element 10 can each have differently shaped profiles here. Regardless of the corresponding diameter of the respective wire D, the necessary clamping force F can still be produced. All the profiles have the property of increasing the friction and/or the clamping force between the wire and the clamp element 10. It is thus possible to apply different wires with different diameters using one and the same surgical instrument 1.

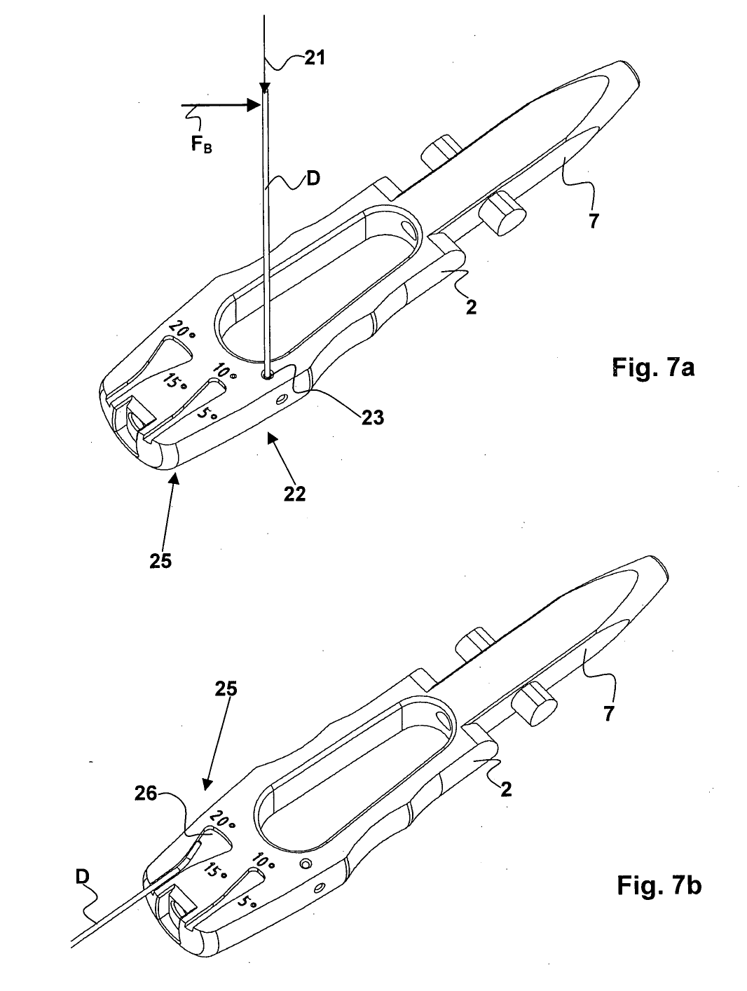

In another preferred illustrative embodiment, the receiving element 2 comprises a device 22 (FIG. 7a). This comprises a bore 23 into which the wire D to be worked is inserted in arrow direction 24. As soon as the corresponding position is reached, a force F_{B} acts on the outer circumference of the wire and thus causes the bending. In particular, this device 22 serves to bend the free end of the surgical wire D in order to facilitate insertion into a bore inside a bone.

To test whether the desired angle of bending of the free end of the wire D is also achieved, a control area 25 is provided, as is shown in FIGS. 7a and 7b. This control area 25 is characterized by having different segments for controlling the angle of bending. For example, a control gage 26 is thus shown in which the wire D, as is illustrated in FIG. 7b, is inserted into the control gage 26. When the outer circumference of the wire D bears on the contour of the control gage 26, the defined angle, here 20 degrees, is reached. Further control gages can be provided.

The surgical instrument 1 according to the invention thus has many advantages. One of the main advantages is that it can be operated with one hand, by virtue of the grip element 4 and the design of the latter. The grip element 4 thus becomes an extension of the surgeon's arm.

By means of the arrangement of the bearing point 6 and bearing elements 9 of the clamp part 3 in relation to the receiving element 2, considerable clamping forces can be achieved, since the lever action multiplies.

The surgical instrument is easy to dismantle and thus easy to clean and also, correspondingly, to sterilize.

The further embodiments comprise preferred bending means with which the free end of the wire can be correspondingly provided with an insertion angle. One and the same surgical instrument is suitable for receiving surgical wires, in particular Kirschner wires.

### LIST OF REFERENCE SIGNS

1 surgical instrument
2 receiving element
3 clamp part
4 grip element
5 grip part
6 bearing point
7 guiding and positioning element
8 grip part
9 bearing element
10 clamp element
11 recess
12 arrow direction
13 arrow direction
14 arrow direction
15 arrow direction
16 bore
17 limit stop
18 start area
19 arrow direction
20
21 arrow direction
22 bending device
23 bore
24 arrow direction
25 control area
26 control gage
27 spring element
28 clamp surface
29 pin
30 recess
D wire
Du outer circumference of wire D
F force (clamping force)
K bone
FB bending force

1. A surgical instrument for implanting a wire, preferably into a bone, consisting essentially of
a receiving element at least for receiving the wire to be applied,
a clamp part at least for clamping the wire,
a grip element at least for holding the surgical instrument,
wherein
the grip element consists of two grip parts, the receiving element comprising the first grip part, and the clamp part comprising the second grip part,
the receiving element has a recess for at least partial support of the wire,
the clamp part is mounted on the receiving element, so as to be able to move in rotation about a bearing point, and comprises a clamp element which engages in the recess and is designed in such a way that, when the grip parts are pressed together, the clamp part presses the wire against the support.

2. The surgical instrument as claimed in claim 1, wherein the clamp part is coupled to the receiving element in the manner of pliers.

3. The surgical instrument as claimed in claim 1, wherein bearing points in the shape of pins are provided in the receiving element and form a connection for the bearing element of the clamp part.

4. The surgical instrument as claimed in claim 3, wherein the pins are connected integrally to the receiving element.

5. The surgical instrument as claimed in claim 1, wherein the bearing elements of the clamp part are designed as eyelets and have openings to one side, such that they can be clipped onto the bearing point of the receiving element.

6. The surgical instrument as claimed in claim 5, wherein the bearing elements are designed in one piece with the clamp part.

7. The surgical instrument as claimed in claim 1, wherein the clamp part has a clamp element with a clamp surface which, to provide clamping, interacts with the wire and the support.

8. The surgical instrument as claimed in claim 7, wherein the clamp part and the clamp element form a one-piece component.

9. The surgical instrument as claimed in claim 1, wherein the receiving element comprises a guiding and positioning element which extends away from the support and has an inner bore for receiving the wire.

10. The surgical instrument as claimed in claim 1, wherein the receiving element comprises a bending device.
